(19) United States
(12) Patent Application Publication
(10) Pub. No.: US 2022/0175366 A1
(43) Pub. Date: Jun. 9, 2022

(54) Stitching Anchor Nail and System As Well As Implantation Method Thereof

(71) Applicant: Intai Technology Corp., Taichung City (TW)

(72) Inventors: Hui-Ping HUANG, TAICHUNG CITY (TW); Ju-Yi HO, TAICHUNG CITY (TW); Hong-Zhe CHIU, TAICHUNG CITY (TW); Li-Yen HOU, TAICHUNG CITY (TW); Tsai-Feng CHANG, TAICHUNG CITY (TW)

(21) Appl. No.: 17/141,838

(22) Filed: Jan. 5, 2021

(30) Foreign Application Priority Data
Dec. 4, 2020 (TW) 109142723

(51) Int. Cl. A61B 17/04

(52) U.S. Cl. CPC A61B 17/0401; A61B 2017/0441; A61B 2017/0414

(57) ABSTRACT

A stitching anchor nail includes an anchor body in a shape of straight tube, a non-circular blade jack formed in and through an axis portion of the anchor body along an axis direction thereof, a thread-in structure at a rear-end part of the anchor body, and a spiral tooth structure formed on a side surface of the anchor body and extending from a rear-end part toward a front end of the anchor body along a spiral direction. The spiral tooth structure is formed with a tooth-in entrance at a front end thereof and has a cross section in a shape of a trapezoid having an included angle ranging from 45 to 55 degree between two side edges of the trapezoid and has a tooth top in a width ranging from 0.4 to 0.7 mm.

### CROSS-REFERENCE TO RELATED APPLICATION

This application claims priority to Taiwan Application Serial Number 109142723, filed Dec. 4, 2020, which is herein incorporated by reference.

### BACKGROUND

### Technical Field

This invention relates to a medical device, and in particular to a stitching anchor nail being implanted into a bone in a surgery and a system thereof as well as an implantation method thereof.

### Description of Related Art

An anchor nail is a surgical instrument applied to stitch soft tissues such as ligaments. In such a stitching surgery, the anchor nail is used to secure one end of a stitching thread to a bone. After the soft tissue is stitched with the thread, another anchor nail is used to tighten and secure the other end of the thread. In order to implant the anchor nail into the bone, the current practice uses a kind of instrument to chisel out a guiding hole in the bone, and then puts a driving rod into the anchor nail and turns the driving rod so as to drive the anchor nail into the bone along the guiding hole. However, there are some problems with the existing practices mentioned above.

First, in existing anchor nails, a spiral tooth around the anchor nail is in a triangular shape. When the anchor nail is screwed into the bone by means of the spiral tooth, an area of contacting the bone with the anchor nail is rather small and a completed structure after such a screwing is less stable. The anchor nail thus cannot closely engage with the bone. Moreover, because the anchor nail is fixed into the guiding hole by the way of the spiral tooth, there is a necessity to chisel out a guiding hole having a big diameter in the bone before screwing the anchor nail thereinto. Such an operation will seriously damage the tissue around the bone and is not good for post-surgery healing. Moreover, the firmness of the anchor nail on the bone is also poor, and the step to change the chiseling instrument and driving rod also make surgery more cumbersome and time-consuming.

### SUMMARY OF THIS INVENTION

Since the conventional anchor has drawbacks comprising a un-firmness structure when it is fixed into the bone, and a need to chisel out a big hole in the bone in advance which in turn may damage the tissue around the bone and result of a poor healing, the present invention thus proposes a design which constitutes a big contact area by a new spiral tooth structure around the anchor nail and forms a tooth-in entrance at a front-end of the spiral tooth, so as to obtain benefits such as firming the screw of the anchor nail into the bone and reducing damage to the tissue around the bone during screwing.

For the above purpose, according to one aspect of the present invention, the present invention proposes a stitching anchor nail comprising:

an anchor body in a shape of straight tube having a front end and a rear end;

a non-circular blade jack formed in and through an axis portion of the anchor body along an axis direction of the anchor body;

a thread-in structure at a rear-end part of the anchor body; and,

a spiral tooth structure formed on the side surface of the anchor body and extending from the rear-end part forward the front end of the anchor body along a spiral direction;

wherein, the spiral tooth structure is sharpened and narrowed down at the front end to form a tooth-in entrance, has a cross section in a shape of a trapezoid having an included angle ranging from 45 to 55 degree between two side edges of the trapezoid, and has a tooth top being defined by a top edge of the trapezoidal cross section and having a width ranging from 0.4 to 0.7 mm.

Preferably, the spiral tooth structure has a height ranging from 0.6 to 0.9 mm and has a tooth bottom defined by a bottom edge of the trapezoidal cross section and having a width ranging from 1.1 to 1.3 mm, and wherein a ratio of the tooth top to the tooth bottom in width size is 0.5.

Alternatively, the cross section of the spiral tooth structure has an included angle at 50 degree between two side edges of the trapezoidal cross section, the tooth top has a height of 0.7 mm and a width of 0.6 mm, and the tooth bottom has a width of 1.2 mm.

Furthermore, the blade jack is in a shape of a polygon having a center at the axis of the anchor body and is formed with at least three blade corners contained in a same hypothetical circular track at the front end of the anchor body, and wherein the tooth-in entrance is located at a position away from one of the blade corners by a lag angle ranging from 10 to 15 degree along a spiral direction so that the tooth-in entrance keeps behind the blade corner when rotates.

According to another aspect of the present invention, for the above purpose, this invention also provides a stitching anchor system, for being fixed into a bone, comprising:

a stitching anchor nail as defined in claim 1; and

a driving blade formed by a rod having a shape corresponding to the blade jack in cross section, and provided with a tip end in a front end thereof;

wherein the driving blade is able to be inserted through the blade jack in the stitching anchor nail and the tip end protrudes out of the blade jack, and wherein the driving blade force the stitching anchor nail to rotate when it is rotated so as to force the stitching anchor nail to be screwed into the bone by means of the spiral tooth structure.

Preferably, the length of the driving blade is a double of the length of the stitching anchor nail and the driving blade is able to be inserted through the blade jack in the stitching anchor nail with its rear part, and out of the blade jack with its front part.

Furthermore, the blade jack is in a shape of a polygon having a center at the axis of the anchor body and is formed with at least three blade corners contained in a same hypothetical circular track at the front end of the anchor body, and wherein the tooth-in entrance is located at a position away from one of the blade corners by a lag angle ranging from 10 to 15 degree along a spiral direction so that the tooth-in entrance keeps behind the blade corner when rotates.

When the stitching anchor nail or stitching anchor system according to this invention is used, the driving blade which accommodates the blade jack will be inserted through the blade jack of the stitching anchor nail. Because the blade jack of the stitching anchor nail is a non-circular shape in cross section, the driving blade accommodating the stitching anchor nail is also a non-circular shape in cross section and is formed with several protruded blades around the side edge thereof.

By means of the above, when the driving blades is rotated, it can drive the stitching anchor nail rotate and enlarge a radius of chiseled hole by the rotation of the protruded blades so as to form a guide hole sufficient to allow a screw-in of the spiral tooth structure and to allow the spiral tooth structure to continuously screw into the guide hole chiseled by the driving blade in advance, starting from the tooth-in entrance at front end thereof. The whole volume of the anchor body can be reduced due to a reduction of the blade jack, and the physician can screw the stitching anchor nail into the bone in one operation. It not only omits a trouble of changing instruments to chisel a hole and to screw the anchor nail, but also reduce a damage to peripheral tissue around the bone during an implantation of the stitching anchor nail into the bone, and thus has benefit to later healing of tissue wounds.

Furthermore, because the spiral tooth structure formed around the anchor body in the present invention, in comparison to a triangular spiral tooth, has a wider included angle between two side walls and a wider width for the tooth top, the spiral tooth structure is closer to a structure of square tooth. Thereby, when the stitching anchor nail is screwed into the bone, it can contact and bite the bone with a larger area and thus can obtain a more secured and stable screw fixing. Thereafter, by using the thread-in structure at the rear-end part to tie a thread into a space between the spiral tooth structure and the bone, the stitching thread for surgery is thus fixed.

For other operating method, the present invention may also only chisel out a puncture hole in the bone for a minimum need by the driving blade and then pull the driving blade back to a position where only the tip end exposures out of the front end of the stitching anchor nail. Next, the driving blade is rotated to force the stitching anchor nail to rotate simultaneously so that the spiral tooth structure starts to rotate and screws into, by way of the tooth-in entrance, the puncture hole. Finally, the driving blade is pulled out of the stitching anchor nail completely, thereby completing the implantation operation of the stitching anchor nail. By means of this kind of operation for implantation, it can make a smallest damage to the peripheral tissue around the bone meanwhile chisel out more bone around the puncture hole during a screw of the spiral tooth structure into the puncture hole, which makes the fixing structure of implanting the stitching anchor nail into the bone B tighter and more stable.

### BRIEF DESCRIPTION OF THE DRAWINGS

The invention will be described with reference to the following drawings, wherein like numerals refer to like elements, and wherein:

FIG. 1 is an explosive diagram of the stitching anchor nail according to a preferred embodiment of the present invention;

FIG. 2 is a three-dimensional diagram of the stitching anchor nail according to the preferred embodiment of the present invention;

FIG. 3 is a three-dimensional diagram from another view angle showing the stitching anchor nail according to the preferred embodiment of the present invention;

FIG. 4 is a cutaway diagram of the stitching anchor nail according to the preferred embodiment of the present invention;

FIG. 5 is an enlarged diagram at a circle portion in FIG. 4 showing the stitching anchor nail according to the preferred embodiment of the present invention;

FIGS. 6A to 6C are diagrams showing a method of using the stitching anchor nail according to the preferred embodiment of the present invention;

FIG. 7 is a three-dimensional diagram of the stitching anchor nail according to another preferred embodiment of the present invention; and,

FIGS. 8A to 8D are diagrams showing another method of using the stitching anchor nail according to another preferred embodiment of the present invention.

### DETAILED DESCRIPTION OF EMBODIMENTS

In the following, with reference to accompanying drawings of embodiments of this invention, technical features and practical effect of this invention will be clearly and completely described as below. Apparently, the embodiments of the invention described below only are a part of embodiments of the invention, but not all embodiments. Based on the described embodiments of the invention, all other embodiments obtained by ordinary skill in the art without creative effort belong to the scope of protection of the invention.

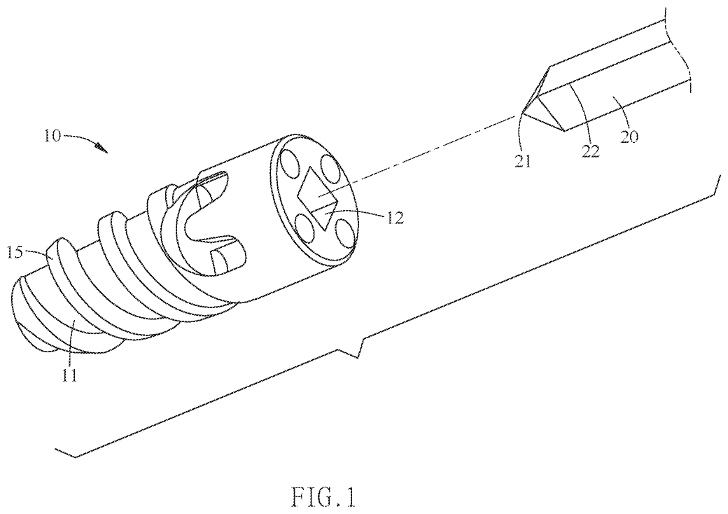

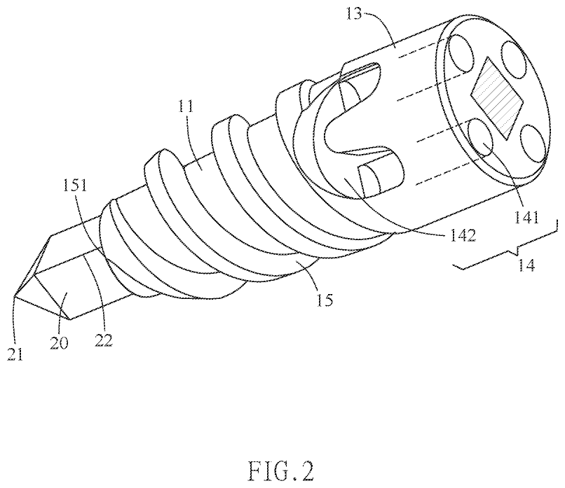

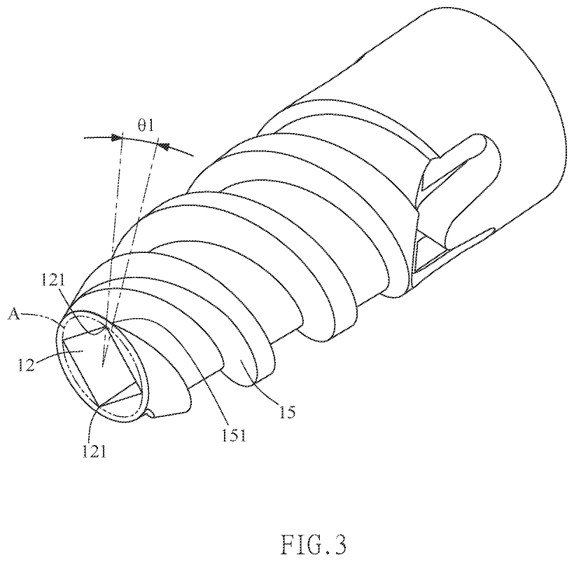

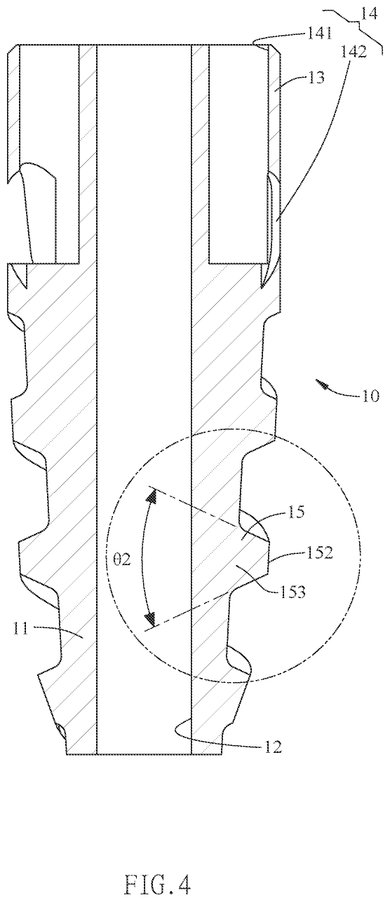

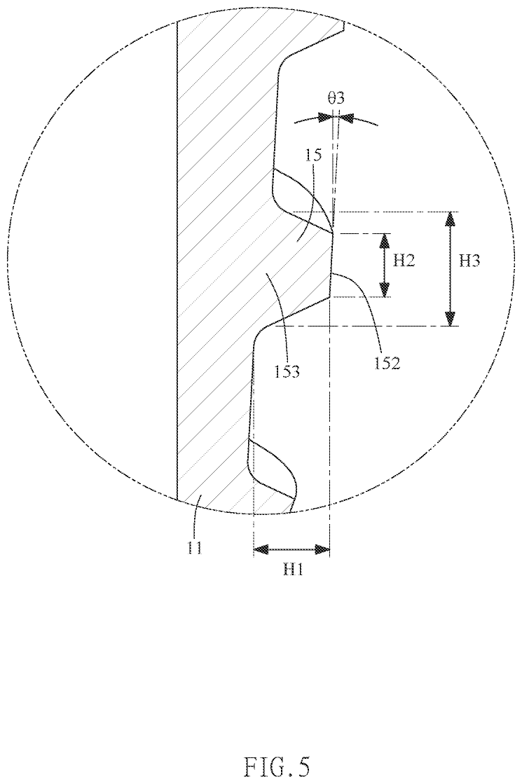

In a preferred embodiment shown in FIG. 1 to FIG. 5, the present invention provides a stitching anchor system, including a stitching anchor nail 10 and a driving blade 20, wherein the stitching anchor nail 10 comprises an anchor body 11 that is in a shape of straight tube having a front end and a rear end.

The stitching anchor nail 10 further comprises a non-circular blade jack 12 formed in and penetrating through an axis portion of the anchor body 11 along an axis direction of the anchor body 11. In this preferred embodiment, the blade jack 12 is in a shape of a square hole having a center on the axis of the anchor body 11. The blade jack 12 is formed with four blade corners 121 contained in a same hypothetic circular track A at the front end of the anchor body 11. The circular track A also have a center on the axis of the anchor body 11.

The stitching anchor nail 10 also comprises a thread installation part 13 in a cylindrical shape at a rear-end part of the anchor body 11. The thread installation part 13 has a diameter greater than that of a front-end part of the anchor body 11. The blade jack 12 penetrates, with its rear portion, through a center portion of the thread installation part 13. The thread installation part 13 comprises a thread-in structure 14 provided with a pair of thread-through holes 141 and a U-shape groove 142 at each of two opposite sides of the thread installation part 13 in relative to the blade jack 12. The thread-through holes 141 extends from a read-end surface of the thread installation part 13 toward the U-shape groove 142 that is formed on a side surface of the thread installation part 13 and corresponds to each pair of thread-through holes 141. The U-shape groove 142 has two ends communicating with the pair of thread-through holes 141 internally.

The stitching anchor nail 10 further comprises a spiral tooth structure 15 that forms on the side surface of the anchor body 11 and extends from the rear-end part forward the front end of the anchor body 11 along a spiral direction. In this preferred embodiment, the spiral direction is in a clockwise direction and the spiral tooth structure 15 is sharpened and narrowed down at the front end, thereby forming a tooth-in entrance 151. The tooth-in entrance 151 is located at a position away from one of the blade corners 121 by a lag angle ø1 along the spiral direction of the spiral tooth structure 15 in relative to the axis of the anchor body 11. In the present preferred embodiment, the lag angle ø1 is 13 degree, so that the tooth-in entrance 151 can allocate at an enter point behind the blade corner 121 when rotates along the spiral direction. The spiral tooth structure 15 has a cross section in a shape of a trapezoid having an included angle ø2 of 50 degree between two side edges of the trapezoid. A top edge and a bottom edge of the trapezoidal cross section of the spiral tooth structure 15 respectively define a tooth top 152 and a tooth bottom 153. In this preferred embodiment, the surface of the tooth top 152 is tilted in a tilted angle ø3 of 2 to 4 degree in relative to the axis of the anchor body 11. A preferred tilted angle ø3 is 3 degree. The tooth top 152 of the spiral tooth structure 15 has a height H1 of 0.7 mm starting from the surface of the anchor boy 11 in a direction perpendicular to the axis of the anchor body 11 and a width H2 of 0.6 mm along a direction parallel to the axis of the anchor body 11. The tooth bottom 153 has a width H3 of 1.2 mm in the direction parallel to the axis of the anchor body 11. The ratio of the tooth top 152 to the tooth bottom 153 in width is 0.5.

The driving blade 20 is formed by a rod having a shape corresponding to the blade jack 12 in cross section. In the present preferred embodiment, the driving blade 20 is a square rod and provided with a tip end 21 in a front end of the driving blade 20. The tip end 21 is a square cone having a tip protruding forward in central portion of the square rod. The driving blade 20 is provided with four corner edges on side surface and each of the corner edge forms a blade 22. In this preferred embodiment, the driving blade 20 has a length slightly longer than the length of the anchor body 11 so that front ends of the four blades 22 around the side surface of the driving blade 20 and the tip end 21 thereof can protrude out of the blade jack 12 when the driving blade 20 is inserted through the blade jack 12 of the stitching anchor nail 10. Since the driving blade 20 is placed inside and engaged with the non-circular blade jack 12 by means of a non-circular matching shape in cross section, the driving blade 20 will drive the stitching anchor nail 10 rotate together when the driving blade 20 is rotated.

In the afore mentioned preferred embodiment, the blade jack 12 and the driving blade 20 are arranged with a corresponding square shape. However, excepting the square shape, the so-call non-circular shape can also be a shape of a star, a triangle, a pentagon or a hexagon and other polygon shapes. Along with a change of the shape, the shape of the cone at the tip end 21 of the driving blade 20 and the number of the blades 22 will also change correspondingly, with a rule that the surrounding multiple blade corners 121 can locate in a same hypothetical circular track A and the lag angle ø1 can be an angle between 10 to 15 degree. Moreover, the height H1 of the spiral tooth structure 15 can be a range from 0.6 to 0.9 mm, the width H2 of the tooth top 152 can be a range from 0.4 to 0.7 mm, the width of the tooth bottom 153 H3 can be a range from 1.1 to 1.3 mm, as long as the ratio of the tooth top 152 to the tooth bottom 153 in width size is 0.5. By means of this setting, the cross section of the spiral tooth structure 15 in the present invention is in a shape closer to a square in relative to a triangle. The thread-in structure 14 can also be a structure having only a pair of thread-through holes 141 at the rear-end surface of the thread installation part 13 that communicates with a U-shape groove 142. Alternatively, the thread-in structure 14 can also be a structure comprising an opening provided in the rear-end part of the anchor body 11 and communicating with the blade jack 12. In such a structure, the driving blade 20 is provided, at side wall, with a straight groove extending through the driving blade 20 and to the front end thereof and exposing outside of the stitching anchor nail 10, so that a stitching thread can be entered from the opening through the straight groove and get out from the front end of the straight groove. By means of this arrangement, the stitching thread can be stranded between the spiral tooth structure 15 and the bone B when the stitching anchor nail 10 is screwed into the bone B.

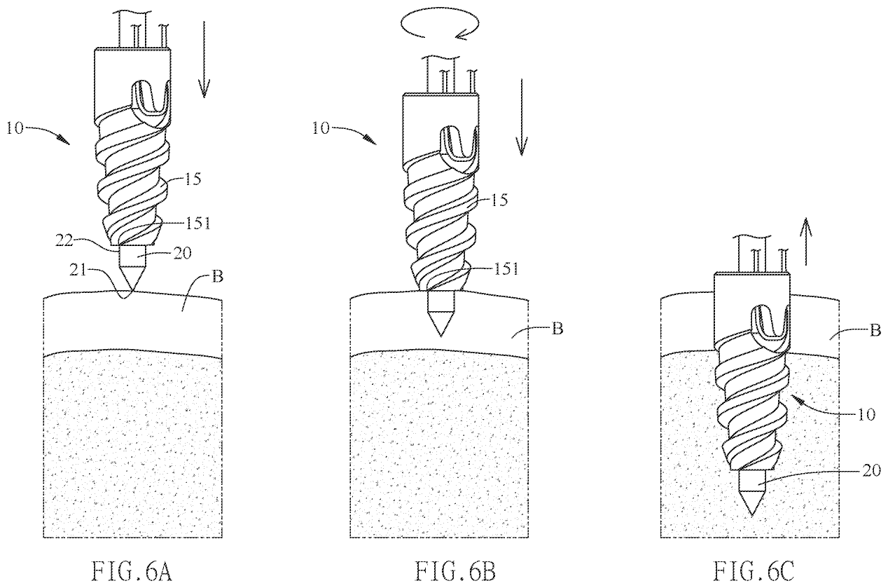

When the stitching anchor system according to the above preferred embodiment of this invention is used, as shown in FIGS. 6A to 6C, the stitching anchor nail 10 is implanted into human body's bone B by cooperating with the driving blade 20. In operation, the rear end of the driving blade 20 is attached to a handheld device, such as a grip, provided for holding by a physician, and then, as shown in FIG. 6B, the physician punches the tip end 21 into a predetermined location in the bone B where the stitching anchor nail 10 will be implanted. Continuously, as shown in FIG. 6B, the physician rotates the driving blade 20 to drill out a guiding hole having a circular cross section. The rotation of the driving blade 20 forces the stitching anchor nail 10 to rotate simultaneously. In the meanwhile, the tooth-in entrance 151 of the spiral tooth structure 15 start to screw into the circular guiding hole that is drilled out by the driving blade 20 in advance. By means of incorporating the arrangement of setting the tooth-in entrance 151 in a position behind one of the blades 22 by a small distance in the progress of contacting the bone B, a progress of screwing the spiral tooth structure 15 into the guiding hole will be smoother. Once the stitching anchor nail 10 is screwed into the bone B with a predetermined depth, as shown in FIG. 6C, an operation of implanting the stitching anchor nail 10 into the bone B is completed just by pulling the driving blade 20 out.

According to the preferred embodiment of this invention, by means of using the driving blade 20, which has an area in cross section smaller than that of the circular track A but can drill out a guiding hole having a size same to the track A, to drive the stitching anchor nail 10, the blade jack 12 in the stitching anchor nail 10 can be a smaller hole, which in turn can reduce the whole volume of the stitching anchor nail 10 and reduce a damage to peripheral tissue due to the implantation of stitching anchor nail 10 into the bone B. Moreover, during the procedure of drilling out the guiding hole by the driving blade 20, the stitching anchor nail 10 is also screwed into the guiding hole simultaneously, thus the physician can avoid a trouble of chiseling out a hole in advance. Furthermore, since the spiral tooth structure 15 has a structure similar to a square tooth that has an included angle of 50 degree between two side walls thereof, a contacting area with the bone B will be bigger than that of a traditional triangle tooth, which in turn results of a more secured and stable fixing effect to the implantation of the stitching anchor nail 10.

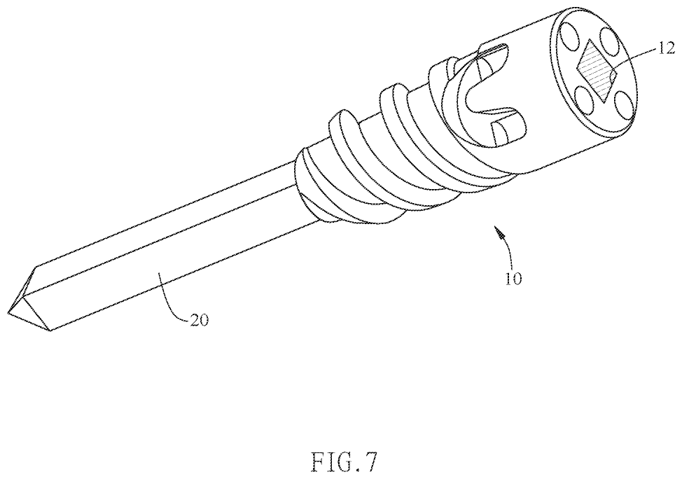

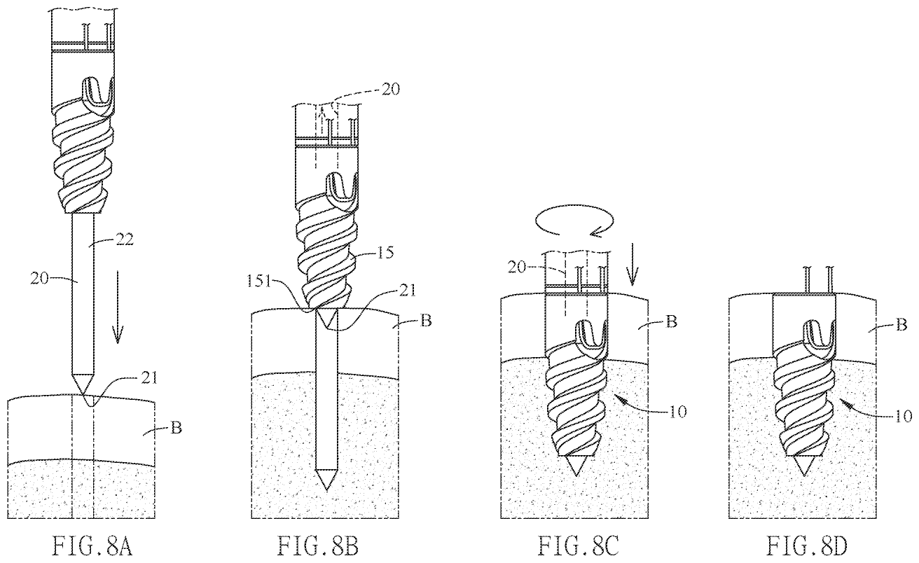

Please refer to FIGS. 7 and 8 which show another preferred embodiment of the present invention. In this embodiment, the length of the driving blade 20 is a double of the length of the stitching anchor nail 10. The driving blade 20 is inserted through the blade jack 12 of the stitching anchor nail 10 with its rear part, and out of the blade jack 12 with its front part.

As shown in FIGS. 8A to 8D, an implantation method of using the stitching anchor system according to another preferred embodiment of this invention comprises the steps of

holding the stitching anchor nail 10 at a rear part of the driving blade 20 first;

punching a device, such as grip, attached to the rear end of the driving blade 20 so as to move the tip end 21 of the driving blade 20 straight forward to chisel out, by using the front part of the driving blade 20, a puncture hole in the bone B that has a length equal to the length of the stitching anchor nail 10;

pulling the driving blade 20 back to a position where only the tip end 21 exposes from the front end of the stitching anchor nail 10;

rotating the driving blade 20 to force the stitching anchor nail 10 to rotate simultaneously so that the spiral tooth structure 15 starts to rotate and screws into, by way of the tooth-in entrance 151 in the front end thereof, the puncture hole chiseled out by a blade of the driving blade 20;

continuously screwing the stitching anchor nail 10 into the bone B with a predetermined depth, wherein the driving blade 20 is only used to drive the stitching anchor nail 10 during this progress and not to chisel out a hole; and,

pulling the driving blade 20 out of the stitching anchor nail 10 completely, thereby completing the implantation operation of the stitching anchor nail 10.

Based upon the implantation method according to another preferred embodiment of the present invention, in addition to the benefit of avoiding a trouble of chiseling out a guide hole in advance and having a more secured and stable fixing effect which are same to the previous preferred embodiment, it can further obtain the benefit of having less damage to the peripheral tissue during the progress of screwing the stitching ancho nail 10 into the bone B and having a better fixing effect. This additional benefit results from a fact that the spiral tooth structure 15 is screwed into the bone B through a hole having a cross section area smaller than that in the previous preferred embodiment, and thus will make less damage to the peripheral tissue meanwhile chisel out more bone around the puncture hole, which makes the fixing structure of implanting the stitching anchor nail 10 into the bone B tighter and more stable and thus creates a better healing effect after surgery.

The present invention has been disclosed in the above with a better embodiment, but the person familiar with this technology should understand that the embodiment is only used to illustrate the present invention, and should not be interpreted as limiting the scope of the present invention. Any change or displacement of the equivalent disclosed by the above embodiments shall be deemed to be covered within the scope of the present invention. Therefore, the scope of protection of the present invention shall be defined by the appended claims of this application for a patent.

1. A stitching anchor nail comprising:
an anchor body in a shape of straight tube having a front end and a rear end;
a non-circular blade jack formed in and through an axis portion of the anchor body along an axis direction of the anchor body;
a thread-in structure at a rear-end part of the anchor body; and,
a spiral tooth structure formed on the side surface of the anchor body and extending from the rear-end part toward the front end of the anchor body along a spiral direction;
wherein, the spiral tooth structure is sharpened and narrowed down at the front end to form a tooth-in entrance, has a cross section in a shape of a trapezoid having an included angle ranging from 45 to 55 degree between two side edges of the trapezoid, and has a tooth top being defined by a top edge of the trapezoidal cross section and having a width ranging from 0.4 to 0.7 mm.

2. The stitching anchor nail as claim 1, wherein the spiral tooth structure has a height ranging from 0.6 to 0.9 mm and has a tooth bottom defined by a bottom edge of the trapezoidal cross section and having a width ranging from 1.1 to 1.3 mm, and wherein a ratio of the tooth top to the tooth bottom in width size is 0.5.

3. The stitching anchor nail as claim 2, wherein the cross section of the spiral tooth structure has an included angle at 50 degree between two side edges of the trapezoidal cross section, the tooth top has a height of 0.7 mm and a width of 0.6 mm, and the tooth bottom has a width of 1.2 mm.

4. The stitching anchor nail as claim 1, wherein the blade jack is in a shape of a polygon having a center at the axis of the anchor body, and is formed with at least three blade corners contained in a same hypothetical circular track at the front end of the anchor body, and wherein the tooth-in entrance is located at a position away from one of the blade corners by a lag angle ranging from 10 to 15 degree along a spiral direction so that the tooth-in entrance keeps behind the blade corner when rotates.

5. The stitching anchor nail as claim 4, further comprises a thread installation part in a cylindrical shape at the rear-end part of the anchor body, wherein the blade jack penetrates through a center portion of the thread installation part, and the thread-in structure is provided with a pair of thread-through holes on at least one of the two opposite sides of a read-end surface of the thread installation part, and provided with a U-shape groove that is formed on a side surface of the thread installation part, corresponds to the pair of thread-through holes, and has two ends communicating with the pair of thread-through holes internally.

6. A stitching anchor system, for being fixed into a bone, comprising:
a stitching anchor nail as defined in claim 1; and
a driving blade formed by a rod having a shape corresponding to the blade jack in cross section, and provided with a tip end in a front end thereof;
wherein the driving blade is able to be inserted through the blade jack in the stitching anchor nail and the tip end protrudes out of the blade jack, and wherein the driving blade force the stitching anchor nail to rotate when it is rotated so as to force the stitching anchor nail to be screwed into the bone by means of the spiral tooth structure.

7. The stitching anchor system as claim 6, wherein the length of the driving blade is a double of the length of the stitching anchor nail and the driving blade is able to be inserted through the blade jack in the stitching anchor nail with its rear part, and out of the blade jack with its front part.

8. The stitching anchor system as claim 6, wherein the blade jack is in a shape of a polygon having a center at the axis of the anchor body, and is formed with at least three blade corners contained in a same hypothetical circular track at the front end of the anchor body, and wherein the tooth-in entrance is located at a position away from one of the blade corners by a lag angle ranging from 10 to 15 degree along a spiral direction so that the tooth-in entrance keeps behind the blade corner when rotates.

9. An implantation method of a stitching anchor system, comprising the steps of
holding a driving blade formed by a rod having a non-circular shape in cross section and provided with a tip end in a front end thereof;
putting the driving blade to be inserted through the blade jack of the stitching anchor nail as defined in claim 1 and keeping the stitching anchor nail on the driving blade,
moving the driving blade straight forward to chisel out a puncture hole in the bone that has a length equal to the length of the stitching anchor nail;
pulling the driving blade back to a position where only the tip end exposes from the front end of the stitching anchor nail; and,
rotating the driving blade to force the stitching anchor nail to rotate simultaneously so that the stitching anchor nail rotates and screws into, by way of the spiral tooth structure, the puncture hole; and
pulling the driving blade out of the stitching anchor nail completely, thereby completing the implantation operation of the stitching anchor nail.

10. The stitching anchor system as claim 9, wherein the length of the driving blade is a double of the length of the stitching anchor nail and the driving blade is able to be inserted through the blade jack of the stitching anchor nail with its rear part, and out of the blade jack with its front part.
